SEQUENCE LISTING (1 sequence)

SEQ ID NO: 1 - DNA, 2888 bp, plasmodium berghei
  gene (1)..(2888)
  ref <308>: DATABASE ACCESSION NUMBER: PBANKA / 1318000
  ref <309>: DATABASE ENTRY DATE: 2015-01-23
  ref <313>: RELEVANT RESIDUES IN SEQ ID NO: (1)..(2888)
atgataaatattggcaatgctattaaaaagacacatttgcctaagaaagaaaaaaaacgtgtctctaaaattttgctaagtaaaaatagaatagaaaataatattttttgtaggaacaataaacttgaaaaagaccacctagaaataaagaatacaaatagaaatatagaatatcaatttcaacctgtaaaaactaataaaacatatagtattaataaaaaggttataagtaataaaataaacaatgaagagttgtatataaaaaaagaaggccacgcaacctttcacatgaatactgacataagagaaaaagatataaataatatattgatgcattctaatgttactacaacaaattttaacagtgaccctattactagtacctcccaaaaatgctcgtctaaaaaaagaatatgtaaacatgctcctataaataaaaaattagattccaataaagaaaatttcgaatcatatacaaataatgcacaaggcgattttggaaactttccccgatttttaagaaaatttgaaaaaataaaaagaaatcccccaattgaacatagtaatgtgcataaaaacgtaggaaaaggtaaaaaaaatgaaatttataagaccaccaaaaattatgaaagcttacaaaataataccaatagaaaaatttatcaaaattgtaaaaaccattatataggaggcatttttaataaaatcgaaaaattaaaaagggataactatcctattatggcaccatcaaattttaaaaataaaaataaattaatcagcaatcattcattaaaatatgaatataccaaggaatggaatgaggaagaaaataaaaataatgataatgcacaaggggatgaatttaaaaaagaagaaaaaaatagttctaataaagatgcttattcaagatatataaagaaaaaaaaatggattaattcagatactattgaaagaattatatccaaaaatagccaacctgatgaacaggtaaaaagaaatccaaacgaaaatattcccaaaaatgatattccaataatagaagtttcacaatggaaagtatttcctgatgttatatcccaattgggtgcacctctcatagaagcatctaattttgaagcaccattacacaagccaacaaaatgtgctgcttcttccaacttttcatacaagttatcagaaaatttttcaggcacactttcaacaaaattagaatgggatgaaaatgaaattttgaaaaatgggaaaactaagaaaaaaaaaaacgaattagacggggttgaagaacatgattatacaattcgaaatacctcgatcacacctgcgtataaacaatataaaataataatcataaaaagcaataacatagaagtagggaactacataataataaaaaatatagggaaaggtacgtttggaaaggtgtgtctaggaatgcatacatatacatacgaaatagttgcaataaaaatattaaataagaaaaaattattacgtataattagttatgataaaatattaagggaaataaaaatccataaaaaaatagatcataatcatatatgtcgtttttatgaagtacatgaaaataaaaataatatatatatgatattagaatatttaggaaatggagatttattaacatatatatgtaaaaatagtaatataaatgaaaacattgcgaaaaggatattataccaattaataagtgctatagaatatttacataaaataaatatagtacatagagatttaaaaccagaaaatatattactggatcataataataatattaaattaattgattttggtttgtcaacaatatatagtaaaaataattttttacaaacatcatgtggatcccctttttatacacctccagaaatactgttaggaaagaaatacaatcctgaattaacagatgtctggtctttaggtataatcctttttttattactcaataaaaaattgccatttaatcataatgacataaataaattatttcaacaaattataaaaggattaatgcaattcgaaccccatgtgtccataaacgcaaaaaaccttattcaaaatatgctcaatgtaaattgtaaaaatagatataatttaaatcagatcaaaaatcatatatggttttctaactataatgctaaaaaggatgtatacttaaattgtcatcaagtatgcaatttaatggattgtaatacttgcatatatatacatataattaaaaccagtaaatattataacaattttttaattaatagaataaataaaatatacaacatagataaaactatgatattaaaagaattaaaaaatgaagaacaaaattgtattaaaacaacatactatcttttgctgaacaaaatggttaagattttatcaaaaactaattacttatattcgcattatatttttataaaaaaagaaacagattcagaaatcttaatttctgatgatgattctcgatctattagtttaaaggataatcatggatcctctaattttacaccatttaataatttaggaattttcaattttttgatctttcaaaaattcaaaaccatgcaccaaaagaataccatgctaatcgaatgaaaaatcgaaacaatggaattagtgaagatatagaacaaattaatacacacaatttattactattgcaacgaaaagaaaagtataatatatttcatataaataaaacacaagtatcaacaaattgttaccattacaaacaaccaaaatgtagattaattaaaaatcaaagaaaagactcgttattttctgacaaacgaggtagtatcaacaatcctaaaaaatgtatcattaaccctgataaagtgcttaaagaaaatatagataaaaagaaaattaacaaaatataa

(19) United States
(12) Patent Application Publication
(10) Pub. No.: US 2019/0054096 A1
(43) Pub. Date: Feb. 21, 2019

(54) METHODS FOR ATTENUATING PARASITE VIRULENCE

(71) Applicant: INSTITUTO DE MEDICINA MOLECULAR, Lisboa (PT)

(72) Inventors: Liliana Mancio SILVA, Subportela, Viana do Castelo (PT); Maria Manuel DIAS DA MOTA, Lisboa (PT)

(21) Appl. No.: 15/764,524

(22) PCT Filed: Sep. 30, 2016
(86) PCT No.: PCT/IB2016/001523

§ 371 Date: Mar. 29, 2018

Related U.S. Application Data
(60) Provisional application No. 62/234,811, filed on Sep. 30, 2015.
(60) Provisional application No. 62/234,808, filed on Sep. 30, 2015.

(51) Int. Cl. A61K 31/60; A61K 31/155; A61K 31/05; A61P 33/00

(52) U.S. Cl. CPC A61K 31/60; A61P 33/00; A61K 31/05; A61K 31/155

(57) ABSTRACT

Pharmaceutical compositions and methods for the treatment of malaria are presented. Such compositions and methods may target energy-sensing pathways of the malaria parasite, Plasmoclium, of parasite host cell, or both. The compositions, in certain aspects of the present invention, target a signalling pathway involving the host AMP-protein activated kinase (AMPK) and/or the parasite AMPK homologue, KIN, which controls parasite replication and virulence.

### CROSS-REFERENCE TO RELATED APPLICATIONS

This application claims priority to U.S. Application No. 62/234,808, filed Sep. 30, 2015; and U.S. Application No. 62/234,811, filed 30 Sep. 2015.

### FIELD OF THE INVENTION

Energy-dependent mechanisms for attenuating proliferation of intracellular parasites.

### BACKGROUND

Parasites require a host in order to survive. As such, they must also have mechanisms to sense and respond to their host's nutritional status, which not only determines nutrient availability but also reflects the quality and viability of the host environment. Plasmodium, the causative agent of malaria, is a rapidly multiplying protozoan parasite that undergoes a complex developmental lifecycle in a vertebrate and mosquito hosts. In the mammalian bloodstream, Plasmodium parasites invade and replicate by schizogony inside red blood cells (RBCs), generating 10-30 new merozoites every 1-3 days, depending on the species. The continuous cycle of new RBC infection by Plasmodium merozoites ultimately leads to the symptoms, morbidity, and mortality associated with malaria, which likely alter the host environment during disease progression. As rapid proliferation requires a rich supply of nutrients, Plasmodium parasites must properly ration such nutrients in order to ensure survival and transmission.

The mechanisms and signalling mechanisms that Plasmodium parasites employ to sense and respond to changes in nutritional needs can be targets for effective chemoprevention of malaria. Thus, the following paragraphs describe novel approaches for treating malaria that exploit the mechanisms used by Plasmodium parasites to rapidly adjust to nutrient fluctuations in a way that has a significant impact on its virulence, thereby affecting the course of infection and onset of disease.

### SUMMARY OF THE INVENTION

The inventions described herein relate to methods and compositions for attenuating proliferation of intracellular parasites. In general, these methods and compositions upregulate. 5′ AMP-activated protein kinase (AMPK) activity, thereby mimicking the natural activation of AMPK caused by fluctuations in (AMP:ATP) ratios in the parasite or host cell.

In various aspects of the invention, methods and compositions of the invention attenuate proliferation of a Plasmodium parasite by activating the parasite's energy-sensing pathways with an AMPK activating agent to mimic the natural role of AMPK activity in inhibiting parasite proliferation under a calorie-restricted diet. In another similar aspect of the invention, methods and compositions of the invention attenuate proliferation of a Plasmodium parasite by activating the host cell's energy-sensing pathways with an AMPK activating agent. The foregoing aspects of the invention are effective for attenuating the proliferation of all Plasmodium species, including all species that are associated with malaria in humans: Plasmodium falciparum, Plasmodium vivax, Plasmodium ovale, Plasmodium malariae, and Plasmodium knowlesi.

### BRIEF DESCRIPTION OF THE DRAWINGS

FIG. 1 exemplifies the attenuation of Plasmodium virulence by calorie restriction (CR). The survival of P. berghei infected C57BL/6 mice on control ad libitum (AL) and CR (n=10 mice/group) is shown. Mice on CR were given daily 60-70% of the food consumed by the control ad libitum (AL) group, for two to three weeks prior and post infection. Mice were infected by i.p. injection of 1×10⁶ erythrocytes infected with P. berghei. Data represents two independent experiments. ****p<0.0001.

FIG. 2 exemplifies a CR diet's alteration of the outcome of a P. berghei liver-stage infection. The graph shows the percentages of infected erythrocytes, a measure of parasitemia, in C57BL/G mice that were infected with freshly dissected sporozoites, and maintained on either AL or CR diets. Data were obtained by flow cytometry analysis of GFP expressing parasites at 72 h.p.i. Data pooled from two independent experiments (≥8 mice/group; each dot represents one animal) is shown, **p<0.01.

FIG. 3 exemplifies a decreased parasite growth rate over the course of a P. berghei blood-stage infection. Data were obtained from blood collected from the tail of C57BL/6 mice that had been infected with 1×10⁶ P. berghei infected erythrocytes. The progression of parasitemia was monitored daily by flow cytometry. Data represents mean±sem from two independent experiments (10 mice/group). ****p<0.0001.

FIG. 4 exemplifies activation of AMPK by CR and CR-mimetic compounds. Western blot analysis of liver homogenates from non-infected mice in AL and DR diets (top panel), and non-infected mice 1 hr after injection of salicylate or vehicle (bottom panel). Lane numbers 1-3 correlate to liver homogenates from individual mice. Representative blot probing with anti-phospho AMPKα (pAMPKα^{T172}), and -actin antibodies.

FIG. 5 exemplifies reduction of P. berghei liver-stage infection by AMPK activating compounds. Relative parasitemia in C57BL/6 mice that were infected with freshly dissected P. berghei sporozoites, and treated with salicylate or metformin, or maintained under control conditions. Salicylate (300 mg/Kg) was administered by i.p. injection once daily, starting one hour before infection, Metformin (500 mg/Kg) was provided in the drinking water for one week prior and during infection, (≥10 mice/group; each dot represents, one mouse). ***p<0.001; ****p<0.0001.

FIG. 6 exemplifies that treatment with AMPK agonists protects P. berghei infected mice from severe malaria, leading to improved survival, C57BL/6 mice were infected and treated as in FIG. 5 (≥10 mice/group; p<0.05).

FIG. 7A exemplifies microscopy analysis and quantification of the number of merozoites per segmented schizont in P. berghei wild-type (wt), Δkin and complemented Δkin (Δkin+kin) parasites. Blood-stage parasites were allowed to mature for 24 hours into schizonts in vitro with medium supplemented with AL and CR sera. KIN is the AMPK homologue in Plasmodium parasites. ****p<0.0001.

FIG. 7B exemplifies reduced parasite replication in the presence of the CR-mimetic compound, salicylate (625 μM; bottom graph). ****p<0.0001.

FIG. 8 exemplifies the global transcriptional changes in P. berghei parasites induced by the CR diet. The graphs represent RNA-sequencing analysis of P. berghei wt and Δkin synchronized parasites collected at 10 hours after re-invasion from AL and CR fed mice (3 mice/group). In the Figure, light grey represents parasite genes that did not change expression in CR compared to AL mice. Highlighted in the darker and darkest shades of grey, respectively, are the induced and repressed genes in CR (absolute fold change greater than 2) for the wild-type parasites (left). The same genes are highlighted in the Δkin graph (right) but with no differential expression. The data suggest that KIN is a caloric nutrient sensor that mediates the transcriptional parasite response to CR.

FIG. 9 exemplifies a dose-dependent effect of CR-mimetic compounds on human P. falciparum blood-stages. Synchronized cultures were set at 0.1% initial parasitemia, treated with the various compounds and analysed by flow cytometry after SYBR Green labelling of parasite DNA at 72 hours or 96 hours post-treatment. Data (mean±sem) was not malted to the untreated control on each experiment (3-5 independent experiments/compound). IC50 values were determined by GraphPad. Prism using non-linear regression variable slope (normalized) analysis. The calculated IC50 values are as follows: salicylate (sal), 1.3 mM; metformin (met), 868 μM; A769662, 71 μM; resveratrol (rsv) 32 μM; SRT1720, 786 nM. Data for rsv and SRT1720 was collected at 72 hours post-treatment, and 96 hours for all the other compounds.

### DETAILED DESCRIPTION

Methods and compositions for attenuating proliferation of intracellular parasites are described herein. More specifically, in various embodiments, methods and compositions of the disclosed invention activate energy-sensing pathways of ether the parasite or the host, or both. Activation of energy sensing pathways, according to the invention, may occur by either direct or indirect cell signalling mechanisms. In various embodiments, methods or compositions of the invention attenuate proliferation of a parasitic organism either directly or indirectly activating 5′ AMP-activated protein kinase (AMPK) in the parasite host cell, which, in turn activates an AMPK-dependent energy-sensing pathway. Thus, it is to be understood, that in venous embodiments, activation of AMPK in a parasite host, or activation of an AMPK homologue in a parasite, mimics the natural upregulation of AMPK activity caused by fluctuations in (AMP:ATP) ratios in the cell, which, in turn leads to decreased replication of the parasite.

It also to be understood, herein, that activation of AMPK signalling in a parasite host cell or activation of signalling of a parasite AMPK homologue, by an AMPK activating agent of the invention may either directly, and/or indirectly, activate a signalling pathway that attenuates parasite proliferation. In various other embodiments, activation of AMPK signalling in a parasite host cell, or activation of signalling of a parasite AMPK homologue, by an AMPK activating agent of the invention simultaneously mediates direct and indirect activation of a signalling pathway that attenuates parasite proliferation. In addition, the methods of the invention accommodate all stages of a parasite life cycle.

In various embodiments, a method of the invention attenuates the proliferation of a parasite belonging to the genus, Plasmodium. Therefore, in an embodiment, an AMPK activating agent contacts a host cell, infected with a Plasmodium parasite, with an amount of an AMPK activating agent that is effective for either direct or Indirect activation of an AMPK-, or an AMPK homologue-, dependent signalling pathway that mediates the attenuation of proliferation, or more specifically, the replication, of the Plasmodium parasite. For example, methods of the invention accommodate the attenuation of at least Plasmodium falciparum, Plasmodium vivax, Plasmodium ovale, Plasmodium malariae, and Plasmodium knowlesi. In addition, as indicated above, methods of the invention accommodate the attenuation of a Plasmodium parasite at all stages of its development and life cycle. For example, in various embodiments, a method of the invention attenuates a Plasmodium parasite at its blood stage, whereas, in other embodiments, a method of the invention attenuates a Plasmodium parasite at its liver stage.

As stated above, various embodied methods of the invention accommodate the activations of host AMPK and parasite AMPK-homologues. For example, in various embodiments, a method of the invention attenuates the proliferation of a Plasmodium species by activating the Plasmodium berghei AMPK homologue, such as KIN, a putative serine/threonine kinase, which controls replication and virulence. KIN is encoded by the polynucleotide sequence associated with the Sanger Institute's GeneDB identifier, PBANKA_1318000, and (SEQ. ID. NO. 1). In various embodiments, activation of KIN by an AMPK activating agent, of the invention either directly, or indirectly, activates a parasite signalling pathway that attenuates parasite proliferation. In some embodiments, activation of KIN by an AMPK activating agent activates KIN by mediating the phosphorylation of a highly conserved threonine in the T-loop of the polypeptide that corresponds to amino acid position 616 of the P. berghei KIN polypeptide. It is to be understood, herein, that the methods of the invention encompass the activation of KIN in any Plasmodium species, including all Plasmodium parasites associated with human hosts, such as, but not limited to, Plasmodium falciparum, Plasmodium vivax, Plasmodium ovale, Plasmodium malariae, and Plasmodium knowlesi.

As used herein, an “AMPK activating agent” refers to a compound, or pharmaceutically acceptable salt thereof, or biological agents that directly or indirectly activate AMPK. AMPK activating agents also include active agents that stimulate one or more pathways resulting in the activation of AMPK.

AMPK activating agents accommodated by compositions of the invention, include, but are not limited to, Thienopyridone derivatives (exemplified in WO2009135580, WO2009124636, US20080221088 an EP1754483; Imidazole derivatives (exemplified in WO2008120797, EP2040702; Thiazole derivatives (exemplified in EP1907369); Guanidine and its derivatives, (galegine, biguanide, and thiazolidinedione), and pharmaceutically acceptable salts thereof. Suitable biguanides include buformin, phenformin and metformin, and pharmaceutically acceptable salts thereof.

Suitable natural product-derived AMPK activating agents accommodated by the methods of the invention include, but are not limited to, Thiazolidinones, (including ciglitazone, MCC-555, rivoglitazone, troglitazone, rosiglitazone, and pioglitazone, and pharmaceutically acceptable salts thereof); Adiponectin; Leptin, Ciliary Neurotrophic Factor (CNTF), Ghrelin; Salicylate, alpha-lipoic acid, alkaloids, and bitter melon extracts.

Other suitable AMPK activating agents include plant polyphenols such as, resveratrol, nootkatone, cucurbitane triterpenoid, momordicoside A, nectandrin B, obovatol, glabridin, damulin B, quercetin, ginsenoside, curcumin, berberine, epigallocatechin gallate, theaflavine, hispidulin, and pharmaceutically acceptable salts thereof.

The invention also accommodates the use of pharmaceutical compositions to activate either parasite or host cell-signalling pathways that mediate a reduction in the parasite's replication rate, thereby providing time and opportunity for the host to combat it. Therefore, methods of the present invention are also directed to the provision of effective pharmaceutically active agents suited for short and long-term prophylaxis and therapeutic treatment.

The term “treatment” as used herein means curative/therapeutic treatment and prophylactic treatment. The terms “curative and therapeutic” as used herein means efficacy in restoring health by curing a disease, e.g., malaria, which has already arisen. Whereas, the term “prophylactic” or “prevention” as used herein means the prevention of the onset or recurrence of the malaria, aimed at maintaining health by preventing ill effects that would otherwise. The term “subject” as used herein refers to a human or non-human animal, including an animal.

In various embodiments, a method of reducing or inhibiting the growth of a Plasmodium species comprises contacting the species with an effective amount of a Plasmodium KIN or AMPK activating compound, or a pharmaceutically acceptable salt thereof, or compositions comprising the same. In such embodiments, the KIN or AMPK activating compound, or a pharmaceutically acceptable salt thereof, or compositions comprising the same, is described above as a KIN or AMPK activating compound or composition comprising the same.

In yet other embodiments, a method of reducing or inhibiting the growth of a Plasmodium species comprises contacting the parasite host cell with an effective amount of an AMPK activating compound, or a pharmaceutically acceptable salt thereof, or compositions comprising the same. In such embodiments, the AMPK activating compound, or a pharmaceutically acceptable salt thereof, or compositions comprising the same, is described above as an AMPK activating compound or composition comprising the same.

Another aspect of methods of treating malaria according to the present invention is that the treatment, (i.e., a pharmaceutical composition described herein), can impair Plasmodium growth in either the parasite's liver or blood growth stages, thus allowing the active agent to target the parasites more specifically. Therefore, in certain embodiments, a treatment for malaria according to the invention impairs Plasmodium growth at the liver stage of development, whereas, in other embodiments, it impairs Plasmodium growth at the blood stage of development, or both.

A therapeutically effective amount of pharmaceutical composition of the present invention will depend upon a number of factors, including biological activity, mode of administration, frequency of treatment, type of concurrent treatment, if any, age, body weight, sex, general health, severity of the Plasmodium infection to be treated, as well as appropriate pharmacokinetic properties. In various embodiments, a method of treatment according to the present invention effectively treats Plasmodium infections that are originated by the species: Plasmodium falciparum, Plasmodium vivax, Plasmodium ovale, Plasmodium malariae, and Plasmodium knowlesi.

Administration and Dosage Forms: In various embodiments of the invention, a pharmaceutically active AMPK activating agent is included within a pharmaceutical composition. for example, in certain such embodiments, a pharmaceutical composition comprises metformin, a salicylate, or a resveratrol. In some further such embodiments; a single pharmaceutical composition comprises at least two pharmaceutically active AMPK-activating agents. As used herein, the term “pharmaceutical composition” refers to a solid or liquid composition, a pharmaceutically active ingredient (e.g., metformin) and at least a carrier, diluent, or excipient, where none of the ingredients is generally biologically undesirable at the administered quantifies.

The pharmaceutical compositions of the invention may be prepared by methods known in the pharmaceutical formulation art, for example, see Remington's Pharmaceutical Sciences, 22nd Ed., (Pharmaceutical Press, 2012), which is incorporated herein by reference. In a solid dosage form, a compound of the invention may be admixed with at least one pharmaceutically acceptable excipient such as, for example, sodium citrate or dicalcium phosphate or (a) (a) fillers or extenders, such as, for example, starches, lactose, sucrose, glucose, mannitol, and silicic acid, (b) binders, such as, for example, cellulose derivatives, starch, aliginates, gelatin, polyvinylpyrrolidone, sucrose, and gum acacia, (c) humectants, such as, for example, glycerol, (d) disintegrating agents, such as, for example, agar-agar, calcium carbonate, potato or tapioca starch, alginic acid; croscarmellose sodium, complex silicates, end sodium carbonate, (e) solution retarders, such as, for example, paraffin, (f) absorption accelerators, such as, for example, quaternary ammonium compounds, (g) wetting agents, such as, for example, cetyl alcohol, and glycerol monostearate, magnesium stearate and the like (h) adsorbents, such as, for example, kaolin and bentonite, and (i) lubricants, such as, for example, talc, calcium stearate, magnesium stearate, solid polyethylene glycols, sodium lauryl sulfate, or mixtures thereof. In the case of capsules, tablets, and pills, the dosage forms may also comprise buffering agents.

Pharmaceutically acceptable adjuvants known in the pharmaceutical formulation art may also be used in the pharmaceutical compositions of the invention. These include, but are not limited to, preserving, wetting, suspending, sweetening, flavoring, perfuming, emulsifying, and dispensing agents. Prevention of the action of microorganisms may be ensured by inclusion of various antibacterial and antifungal agents, for example, parabens, chlorobutanol, phenol, sorbic acid, and the like. It may also be desirable to include isotonic agents, for example, sugars, sodium chloride, and the like. If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

Solid dosage forms as described above may be prepared with coatings and shells, such as enteric coatings and others, as is known in the pharmaceutical art. They may contain pacifying agents, and can be of such composition that they release the active compound or compounds in a certain part of the intestinal tract in a delayed manner. Non-limiting examples of embedded compositions that may be used are polymeric substances and waxes. The active compounds may also be in microencapsulated form, if appropriate, with one or more of the above-mentioned excipients.

Suspensions, in addition to the active compounds, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like. Liquid dosage forms may be aqueous, may contain a pharmaceutically acceptable solvent as well as traditional liquid dosage form excipients known in the art, which include, but are not limited to, buffering agents, flavorants, sweetening agents, preservatives, and stabilizing agents.

### EXAMPLES

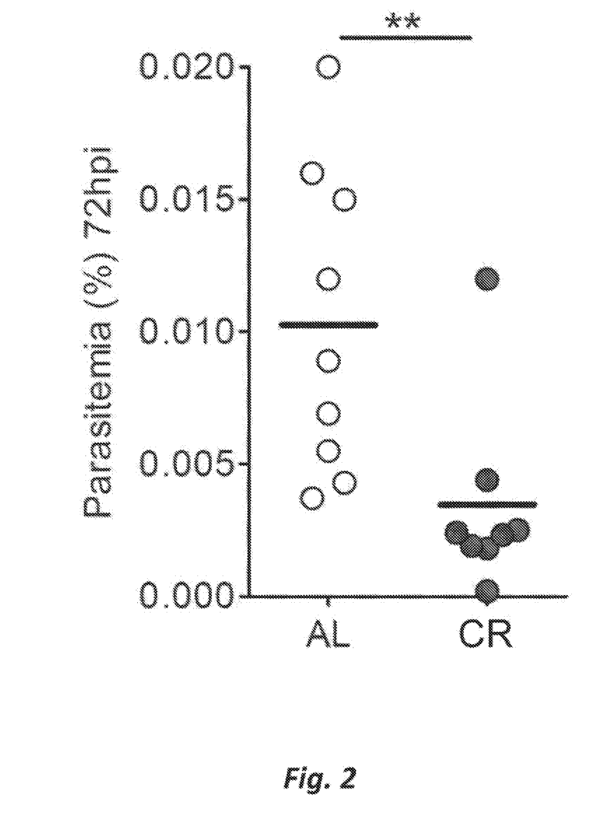

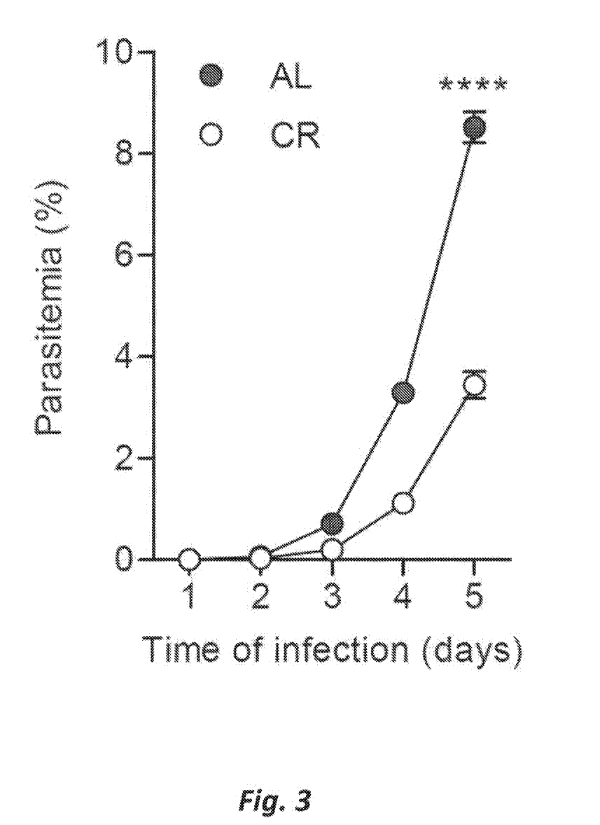

Example 1. Host diet affects survival and parasite load. C57BL/6 mice (age 5-8 weeks; weight 20-28 g) were either allowed free access to water and food, or placed on calorie restriction (CR). Mice on CR were daily given 60-70% of the food consumed by the control group ad libitum (AL). Food intake in both groups was measured daily and body weights at least 3 times a week. Upon reaching 15-20% weight loss, the daily food allotted to CR mice or rats was adjusted to stabilize the lower body weights for the remainder of the experimental period. The mice were infected by intradermal (i.d.) injection of 5×10³ freshly dissected P. berghei sporozoites (FIG. 2) or by intraperitoneal (i.p.) Injection of 10⁶ P. Berghei-infected erythrocytes, which were obtained by prior passage in the C57BL/6 mice (FIG. 3). The mice were housed three to five per cage.

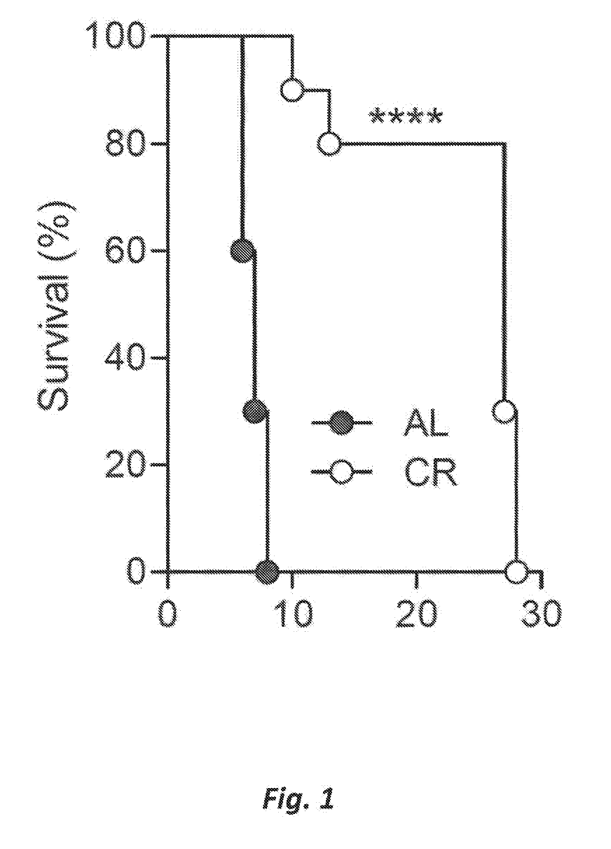

Infection resulted in a significant reduction of liver stage infection, and suppressed asexual blood stage parasitemia in CR animals relative to the control AL mice. After 5-6 days of P. berghei blood-stage infection, AL fed mice lose weight and succumb from severe disease, whereas CR mice survive with little weight change and slow increase in parasitemia overtime. See FIGS. 1-3. Parasitemia was determined by flow cytometry analysis (BD FACSCAlibur200 or BD LSRFortessa®) of one drop of tail blood in PBS from C57BL/6 mice that were infected with GFP-expressing P. berghei parasites, and maintained on either AL or CR diets. Analysis on FlowJo® followed. See FIGS. 2 and 3. The number of acquired total events ranged from 100-200 thousand. Infected erythrocytes were selected based on their size by gating first on FSC and SSC and, subsequently, on FITC (green) and PE (red) channels. The GFP-expressing parasites were detected in the FITC channel. False GFP positive ceils (erythrocyte's auto-fluorescence) were eliminated by plotting FITC against PE.

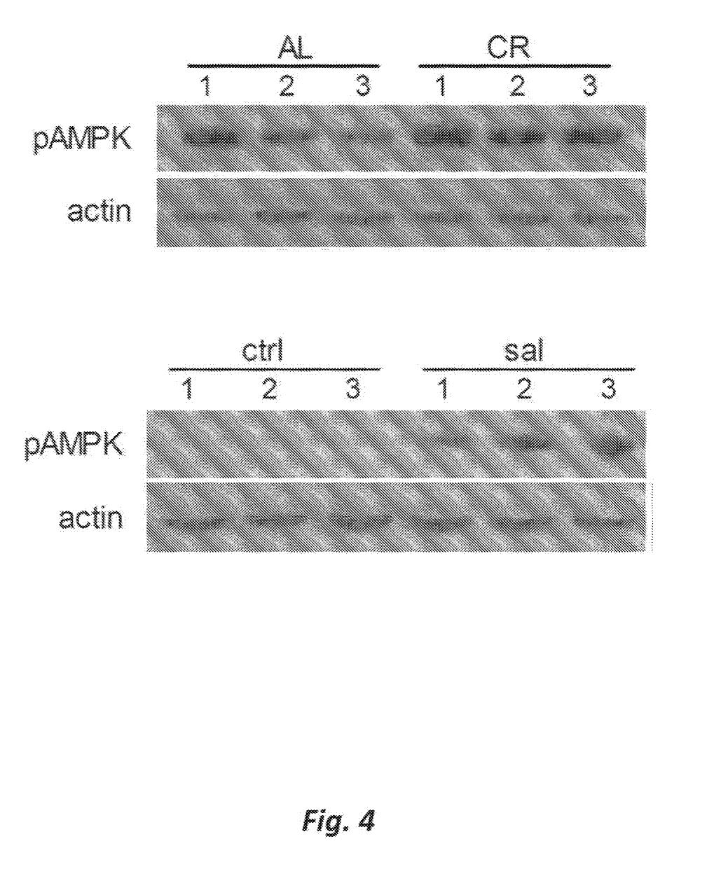

Example 2. AMPK activating compounds mimic CP-mediated activation of AMPK. Western blot analysis of AMPK phosphorylation in liver homogenates from non-infected C57BL/6 mice in AL end DR diets, and non-infected mice one hour after injection of salicylate or vehicle. See FIG. 4. Livers were homogenized in ice-cold lysis buffer (50 mM HEPES, 150 mM NaCl, 10 mM NaF, 1 mM Sodium pyrophosphate, 0.5 mM EDTA, 1 mM DTT, 1% triton, 1 mM Na₃VO₄, 250 mM Sucrose, protease inhibitor cocktail and phosphatase inhibitors). Total protein content in each homogenate was measured by Bradford Assay (Bio-Rad), according to manufacturer's instructions. 50 μg of total liver lysates were resolved on either 8% SDS-PAGE or Any kD™ Mini-PROTEAN® precast gels (Bio-Rad) and transferred to a nitrocellulose membrane using standard wet transfer with 1× Tris-Glycine buffer containing 20% methanol for 2 hr at 100 V constant or were transferred using iBlot® gel Transfer Stacks (ThermoFisher). Membranes were blocked in 5% BSA TBSTween 0.2% for 1 hr at room temperature and incubated with primary antibodies overnight at 4° C.

pAMPK was detected using rabbit anti-phospho-AMPKα T172 (mAb 40H9.1:1000, Cell Signaling Technology) (incubation for overnight at 4° C). Anti-actin (1:1000, Sigma-Aldrich A2066) rabbit antibody was used as loading control (incubation for 1 hr at room temperature). Horseradish peroxidase (HRP)-conjugated goat anti-rabbit IgG, Fc fragment specific and HRP-conjugated goat anti-mouse IgG, light chain specific (both from Jackson ImmunoResearch) were used as secondary antibodies.

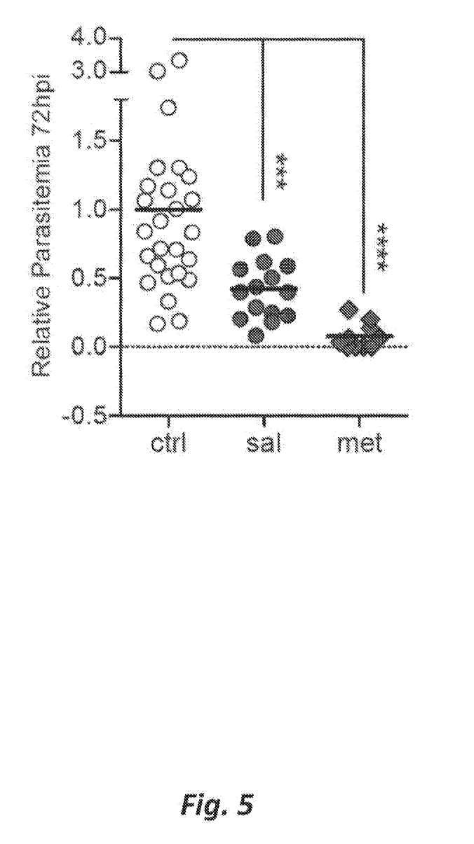

Example 3. Reduction of Plasmodium liver-stage infection by AMPK activating compounds. Relative parasitemia in C57BL/6 mice at 72 hours post infection (h.p.i) with freshly dissected P. berghei sporozoites, was determined by flow cytometry analysis (as described in Example 1) for mice treated with either salicylate or metformin, or maintained under control, untreated conditions. Salicylate (300 mg/Kg, Sigma No. 71945) was administered by i.p. injection once daily, smarting one hour before infection. Metformin (500 mg/Kg, Sigma No D150959) was provided in the drinking water for one week prior to, and during, infection. Results are reported in FIG. 5.

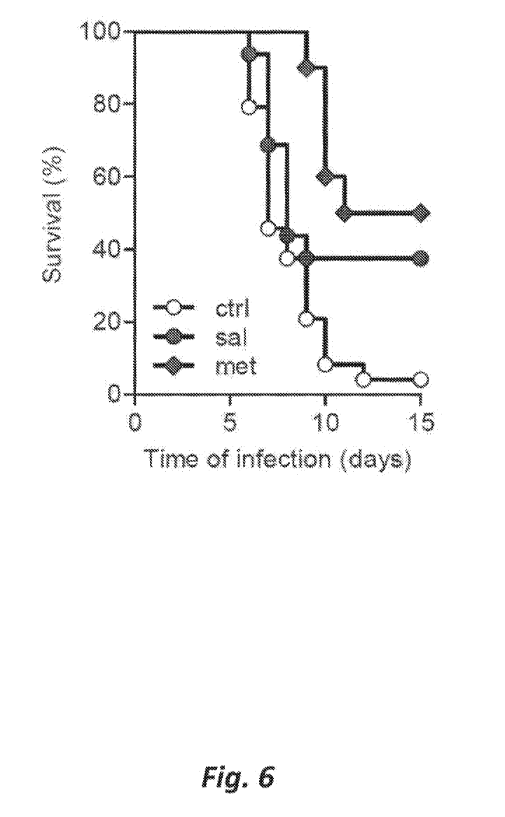

Example 4. Improved survival of parasite-infected mice treated with AMPK agonists. To determine the effect of the AMPK activators, salicylate and metformin, on animal survival over a 15-day period following parasite infection, C57BL/6 mice were infected by i.d. injection of 5×10³ P. berghei sporozoites. Salicylate (300 mg/Kg, Sigma No. 71945) was administered by i.p. injection once daily, starting one hour before infection. Metformin (500 mg/Kg. Sigma No D150959) was provided in the drinking water for one week prior to, and during, infection. Results, showing significant increases in the survival rates of mice that were treated with the AMPK activators are reported in FIG. 6.

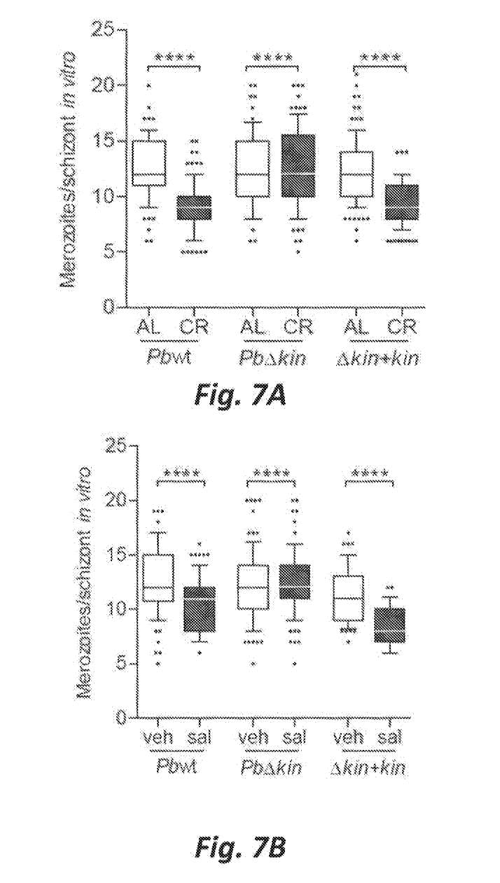

Example 5. The AMPK homologue, KIN, regulates the CR effect in Plasmodium parasites. P. berghei parasite wild-type respond to CR by decreasing the number of daughter merozoites on each cycle. By contract, parasites lacking the kin gene (Δkin) fail to respond to CR and generate merozoite numbers comparable to wild-type parasites, both in AL and CR. The CR effect phenotype can be rescued by reintroducing the kin gene into Δkin parasite line. See FIG. 7A. Similarly, while wild-type and complemented Δkin reduce replication in the presence of the AMPK agonist, salicylate, the Δkin parasites fail to respond to the drug. See FIG. 7B.

P. berghei blood-stage parasites can be maintained in vitro for only one developmental cycle (20-24 h), in which ring forms develop into mature schizonts, without bursting. Infected blood was obtained from in vivo infections containing mainly ring-stage forms at 1-3% parasitemia. Parasites were cultured in RPMI1640 medium containing 25 mM HEPES, 0.05 mg/ml Gentamicin or Penicillin/Streptomycin (all Gibco/Invitrogen), in 96-well plates. Culture medium was supplemented with 25% of mouse serum collected from animals in AL or CR diets. For drug testing, the medium was supplemented with 25% fetal bovine serum. 24 h after incubation at 37° C. in 5% O₂, 5% CO2, 90% N2, blood smears were made and stained with Giemsa. Parasite development and the number of merozoites per schizont were then assessed using light microscopy and image (http://rsbweb.nih.gov/ij/). Only mature schizont (segmenters) with clearly separated merozoites were scored.

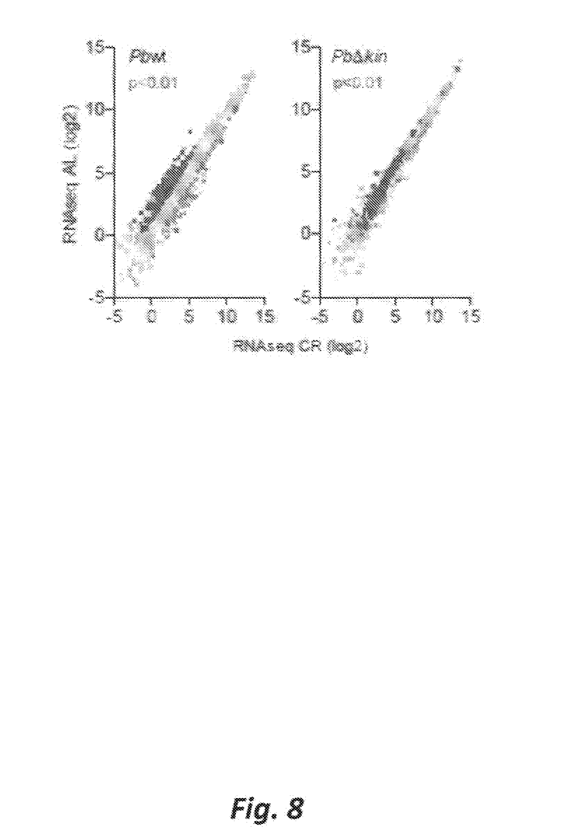

Example 6. Global transcriptional changes in P. berghei parasites induced by the CR diet. RNA-sequencing analysis of P. berghei wild-type and Δkin parasites was performed. The analysis revealed significant differential transcription of approximately 600 genes in the wild-type parasites in CR diet compared to AL, whereas no differential gene expression was observed in Δkin parasites in the two diets. See FIG. 8. These data suggest that KIN is a caloric nutrient sensor that mediates the parasite response to CR.

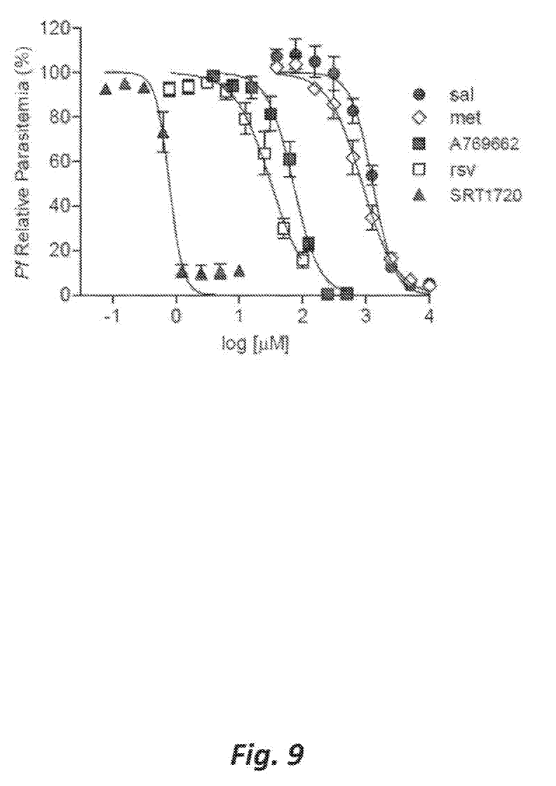

Example 7. Dose-dependent effect of CR-mimetic compounds on human P. falciparum blood-stages. Synchronized cultures of the P. falciparum parasite line, 3D7, were set at 0.1% initial parasitemia, treated with the various compounds and analysed by flow cytometry after SYBR Green labelling of parasite DNA at 72 hours or 96 hours post-treatment. The calculated IC₅₀ values are as follows: salicylate (sal), 1.3 mM; metformin (met), 868 μM; A769662 A769662 (Calbiochem), 71 μM; resveratrol (rsv), 32 μM; SRT1720, 786 nM. Data for rsv and SRT1720 were collected at 72 hours post-treatment, and 96 hours for all the other compounds. See FIG. 9.

P. falciparum 3D7 was continuously cultured in human erythrocytes at 4% hematocrit in RPMI 1640 supplemented with 0.5% Albumax II (Invitrogen), 200 mM Hypoxanthine (Sigma) and 20 μg/ml Gentamicin (Invitrogen). Cultures were maintained at 37° C. in an atmosphere of 5% O₂, 5% CO₂, 90% N₂, and synchronized by consecutive treatments of 5% Sorbitol (Sigma), for dose-response analysis, serial dilutions of the compounds were added to synchronized ring-stages of P. falciparum in 96-well plates. Initial parasitemia was set to 0.1% and hematocrit 2%. Parasite replication and reinvasion were assessed at the indicated time by flow cytometry based on fluorescent labelling of P. falciparum DNA. The SYBR Green I (Invitrogen) stained samples were analysed on a CyFlow® SL Blue or Accuri® BD flow cytometer and the data evaluated using FlowJo software (TreeStar) to determine the percentage of infected erythrocytes. IC50 values were calculated using GraphPad Prism.

1. A method for attenuating proliferation of a Plasmodium organism in a host cell, comprising contacting the host cell with an effective amount of a 5′ AMP-activated protein kinase (AMPK) activating agent to attenuate the proliferation of the Plasmodium organism, wherein the AMPK activating agent activates a host cell AMPK, a Plasmodium AMPK homologue, or a host cell AMPK and a Plasmodium AMPK homologue.

2. (canceled)

3. The method of claim 1, wherein the activation of a host cell AMPK, a Plasmodium AMPK homologue, or a host cell AMPK and a Plasmodium AMPK homologue, decreases Plasmodium parasite replication.

4. The method of claim 1, wherein a Plasmodium infection in an individual is treated.

5. The method of claim 1, wherein the Plasmodium AMPK homologue is KIN.

6. The method of claim 1, wherein Plasmodium parasite proliferation is decreased at either the Plasmodium liver stage or blood stage of infection.

7. The method of claim 1, wherein the AMPK activating agent indirectly activates energy-sensing pathways that are naturally upregulated in nutrient limiting conditions.

8. The method of claim 7, wherein the AMPK activating agent directly activates an energy-sensing pathway.

9. (canceled)

10. The method of claim 1, wherein the AMPK-activating agent is a guanidine, a biguanide, a thiazolidinedione, a salicylate, or a plant polyphenol.

11. The method of claim 10, wherein the AMP-activation agent is a biguanide.

12. The method of claim 11, wherein the biguanide is buformin, phenformin, metformin, or a pharmaceutically acceptable salt thereof.

13. The method of claim 12, wherein the biguanide is metformin or a pharmaceutically acceptable salt thereof.

14. The method of claim 10, wherein the AMPK agent is a salicylate.

15. The method of claim 10 wherein the AMPK activation agent is a plant polyphenol.

16. The method of claim 15, wherein the plant polyphenol is resveratrol, nootkatone, cucurbitane triterpenoid, momordicoside A, nectandrin B, obovatol, glabridin, damulin B, quercetin, ginsenoside, curcumin, berberine, epigallocatechin gallate, theaflavine and hispidulin, or a pharmaceutically acceptable salt thereof.

17. The method of claim 16, wherein the plant polyphenol is resveratrol.

18. The method of claim 4 further comprising a first step of administering the AMPK activating agent is administered to the individual in a pharmaceutical composition, comprising a pharmaceutically acceptable adjuvant, a carrier, a diluent, or a combination thereof.

19. The method of claim 18, wherein the pharmaceutical compositions is in a form suitable for parenteral administration or oral administration.

20. The method of claim 1, wherein the Plasmodium organism is capable of proliferating in a human host cell.

21. The method of claim 20, wherein the Plasmodium organism is Plasmodium falciparum, Plasmodium vivax, Plasmodium malariae, Plasmodium knowlesi, or Plasmodium ovale.
